(19) United States
(12) Patent Application Publication
(10) Pub. No.: US 2022/0287569 A1
(43) Pub. Date: Sep. 15, 2022

(54) System for Detecting Body Temperature with Temperature Compensation

(71) Applicant: Ziweixing Co., LTD., Taipei City (TW)

(72) Inventor: Wan-Tien Chen, Taipei City (TW)

(21) Appl. No.: 17/563,123

(22) Filed: Dec. 28, 2021

(30) Foreign Application Priority Data
Mar. 9, 2021 (TW) 110202499

(51) Int. Cl. A61B 5/01; A61B 5/00; A61B 5/1171; G06V 40/18

(52) U.S. Cl. CPC A61B 5/01; A61B 5/0077; A61B 5/1176; A61B 5/7203; G06V 40/18; A61B 2560/0252; A61B 2562/0271

(57) ABSTRACT

This invention provides a system for detecting body temperature with temperature compensation. The system includes: a first sensor, detecting some objects in an area and performing an identifying procedure; a second sensor, measuring temperatures of those objects and outputting several ambient temperatures and body temperatures, wherein the ambient temperatures and the body temperatures are identified as non-body temperatures and body temperatures by the identifying procedure; and a central processing unit, receiving and averaging the ambient temperatures to produce a current temperature, and reading a reference temperature, comparing the current temperature with the reference temperature to produce a compensation, performing operations for the compensation and the body temperatures to output some actual body temperatures.

### BACKGROUND OF THE INVENTION

### 1. Field of the Invention

The present invention relates to the field of body temperature detection, and more particularly, to a system that can capture face images with precision identification, avoid interference from other non-human heat sources and match body temperature detection with real-time feedback of accurate temperature.

### 2. Description of the Prior Art

The body temperature is constant, but the main reason for the relative instability of the general market-available thermal sensing instruments is that the body surface temperature measured by the subject will change with the ambient temperature, resulting in the body surface temperature being different from the actual body temperature.

The present invention is based on thermal sensing to measure the ambient temperatures many times and then to average them, and further to automatically compensate based on the ambient temperature at that time. This allows the body surface temperature measured by the subject to be closer to the actual body temperature after compensation to improve the accuracy of the temperature measurement.

### SUMMARY OF THE INVENTION

According to a preferred embodiment of the present invention, it provides a system for detecting body temperature with temperature compensation. The system includes: a first sensor, detecting some objects in an area and performing an identifying procedure, wherein the identifying procedure includes facial recognition; a second sensor, measuring temperatures of those objects in the area and outputting several ambient temperatures and body temperatures, wherein the ambient temperatures are identified as non-body temperatures by the identifying procedure, the body temperatures are identified as body temperatures by the identifying procedure; and a central processing unit, receiving and averaging the ambient temperatures to produce a current temperature, reading a reference temperature, comparing the current temperature with the reference temperature to produce a compensation, and performing operations for the compensation and the body temperatures to output some actual body temperatures.

In one embodiment, the first sensor comprises an RGB thermal camera.

In one embodiment, the area comprises 2 to 3 meters.

In some embodiments, the identifying procedure comprises iris recognition.

In one embodiment, the second sensor comprises a non-contact temperature detecting sensor.

In one embodiment, each of the body temperatures further includes several temperatures, the central processing unit only takes the maximum one in the temperatures to operate with the compensation.

In one embodiment, the reference temperature comprises a temperature-set corrected value.

In some embodiments, each of the body temperatures further includes several temperatures, the central processing unit only takes the maximum one in the temperatures to perform a linear compensation operation, including: y=ax+b, wherein y is an actual body temperature, x is the maximum one, a represents a proportional value, and b represents a compensation constant.

In some embodiments, each of the body temperatures further includes several temperatures, the central processing unit only takes the maximum one in the temperatures to perform a non-linear compensation operation, including: \sqrt{y}=ax²+bx+c, wherein y is an actual body temperature, x is the maximum one, a, b represent proportional values, and c represents a compensation constant.

The present invention provides a system for detecting body temperature with temperature compensation. It continuously detects ambient temperatures many times by thermal sensing and gets an average temperature. It measures the average temperature in terms of big data of the ambient temperatures and gets the difference to an original reference temperature to perform compensation to the ambient temperature at that time automatically, so that the body surface temperature measured by the subject is closer to the actual temperature after compensation and the accuracy of the temperature measurement is also improved.

### BRIEF DESCRIPTION OF THE DRAWINGS

The present invention can be more fully understood by reading the following detailed description of the preferred embodiments, with reference made to the accompanying drawings, wherein:

FIG. 1 shows a block diagram according to a preferred embodiment of the present invention; and

FIG. 2 shows a block diagram according to another preferred embodiment of the present invention.

### DETAILED DESCRIPTION OF THE PREFERRED EMBODIMENTS

Some embodiments of the present invention are described in detail below. However, in addition to the descriptions given below, the present invention can be applicable to other embodiments, and the scope of the present invention is not limited by such, rather by the scope of the claims. Moreover, for better understanding and clarity of the description, some components in the drawings may not necessary be drawn to scale, in which some may be exaggerated relative to others, and irrelevant parts are omitted.

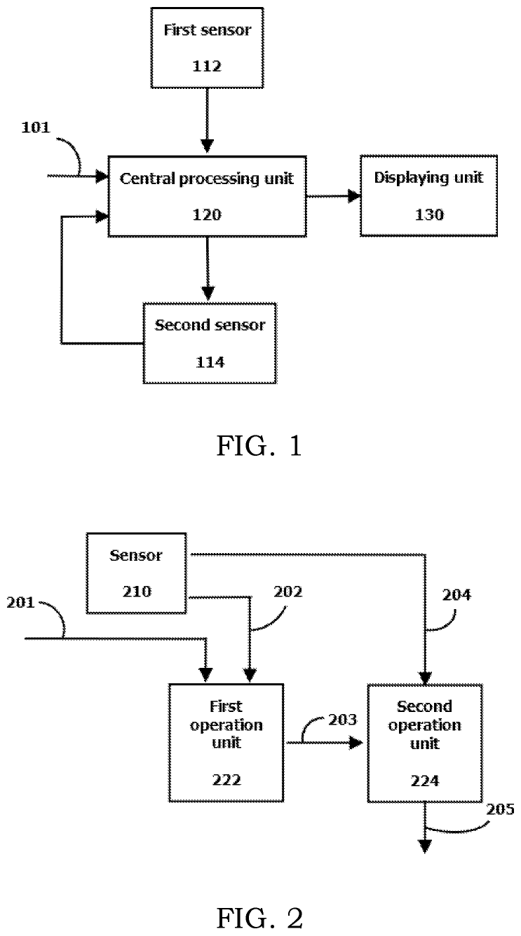

Referring to FIG. 1, a block diagram for a preferred embodiment according to the present invention is illustrated. A first sensor 112 is used to detect several objects in an area and perform an identifying procedure, wherein the area includes 2 to 3 meters around the first sensor 112, and the identifying procedure could include biometric, such as iris recognition, a facial recognition is implemented in the present embodiment, but not limited to. In the present embodiment, the first sensor 112 could include an RGB thermal camera. A second sensor 114 is used to measure several temperatures of those objects in the area and output several ambient temperatures and body temperatures, wherein the ambient temperatures are identified as non-body temperatures by the identifying procedure, the body temperatures are identified as body temperatures by the identifying procedure. In the present embodiment, the second sensor 114 could include a non-contact temperature detecting sensor, such as infrared thermal sensor, but not limited to. In the present embodiment, the ambient temperatures could include the ambient temperatures of multiple points except the temperatures of human bodies which are around the second sensor 114 in 2 to 3 meters and are detected by the second sensor 114. The abovementioned body temperatures could include everybody's temperature which further includes multiple temperatures, that is, when the first sensor 112 identifies the detected object as a human body, the second sensor 114 can measure multiple temperatures for this human body and is not limited to only one temperature measured for one human body.

A central processing unit 120 is used to receive and average the ambient temperatures to produce a current temperature. For example, the central processing unit 120 receives the ambient temperatures of multiple points which are around the second sensor 114 in 2 to 3 meters and are detected by the second sensor 114 to accumulate and average them to produce the current temperature. The central processing unit 120 also reads a data 101, referring to a reference temperature in the present embodiment, wherein the reference temperature includes a temperature-set corrected value. The central processing unit 120 compares the current temperature with the reference temperature to produce a compensation. In the present embodiment, the reference temperature could be a set of data saved in an external/internal non-volatile memory and read by using a lookup table or could be parameter(s) read while a program performs, but not limited to. The central processing unit 120 performs operations for the compensation and the body temperatures to output some actual body temperatures to a displaying unit 130. In one embodiment, the body temperatures could include 5 to 10 people's body temperatures, wherein each body temperature could further include multiple temperatures. The maximum temperature in each body temperature can be seen as the body temperature of each person. Or, in other embodiments, the average temperature in each body temperature can be seen as the body temperature of each person.

In one embodiment, the central processing unit 120 may use the maximum temperature in each body temperature, but not limited to, to perform a linear compensation operation, including: y=ax+b, wherein y is an actual body temperature, x is the maximum one, a represents a proportional value, and b represents a compensation constant. Wherein, a is an average value for the proportions of the maximum temperatures and the actual body temperatures measured by many times. Wherein, b is an average value for the differences of the maximum temperatures and the actual body temperatures measured by many times.

In another embodiment, the central processing unit 120 may use the maximum temperature in each body temperature, but not limited to, to perform a non-linear compensation operation, including: \sqrt{y}=ax²+bx+c, wherein y is an actual body temperature, x is the maximum one, a, b represent proportional values, and c represents a compensation constant. Wherein, a and b are two average values for the proportions of the maximum temperatures and the actual body temperatures measured by many times. Wherein, c is an average value for the differences of the maximum temperatures and the actual body temperatures measured by many times.

By doing so, the system for detecting body temperature with temperature compensation provided by the present invention can detect several body temperatures around 2 to 3 meters, avoid interference from other non-human heat sources, and continuously perform compensation to the ambient temperature at that time automatically, so that the body surface temperature of the subject is closer to the actual temperature after compensation and the accuracy of the temperature measurement is also improved.

Referring to FIG. 2, a block diagram for another preferred embodiment according to the present invention is illustrated. A sensor 210 is used to detect several objects and the surface temperatures thereof in an area, perform an identifying procedure, and output several corresponding ambient temperatures 202 and body temperatures 204. Wherein, the ambient temperatures 202 are identified as non-body temperatures by the identifying procedure, the body temperatures 204 are identified as body temperatures by the identifying procedure. Wherein, the area includes 2 to 3 meters around the sensor 210, and the identifying procedure could include biometric, such as iris recognition, a facial recognition is implemented in the present embodiment, but not limited to. In one embodiment, the sensor 210 could include the first sensor 112 and the second sensor 114 shown in FIG. 1. In one embodiment, the ambient temperatures 202 could include the ambient temperatures of multiple points except the temperatures of human bodies which are around the sensor 210 in 2 to 3 meters and are detected by the sensor 210. The abovementioned body temperatures 204 could include everybody's temperature which further includes multiple temperatures, that is, when the sensor 210 identifies the detected object as a human body, the sensor 210 can measure multiple temperatures for this human body and is not limited to only one temperature measured for one human body.

A first operation unit 222 is used to receive, accumulate, and average the ambient temperatures 202 to produce a current temperature. For example, the first operation unit 222 receives the ambient temperatures of multiple points which are around the sensor 210 in 2 to 3 meters and are detected by the sensor 210 to accumulate and average them to produce the current temperature. The first operation unit 222 also reads a data 201, referring to a reference temperature in this embodiment, wherein the reference temperature includes a temperature-set corrected value. The first operation unit 222 compares the current temperature with the reference temperature to produce a compensation 203. In one embodiment, the reference temperature could be a set of data saved in an external/internal non-volatile memory and read by using a lookup table or could be parameter(s) read while a program performs, but not limited to. Then, a second operation unit 224 performs operations for the compensation 203 and the body temperatures 204, such as addition operation, to output some actual body temperatures. In one embodiment, the body temperatures could include 5 to 10 people's body temperatures, wherein each body temperature could further include multiple temperatures. The maximum temperature in each body temperature can be seen as the body temperature of each person. Or, in other embodiments, the average temperature in each body temperature can be seen as the body temperature of each person. In one embodiment, the first operation unit 222 and the second operation unit 224 could be included in the central processing unit 120 shown in FIG. 1.

In one embodiment, the second operation unit 224 may use the maximum temperature in each body temperature, but not limited to, to perform a linear compensation operation, including: y=ax+b, wherein y is an actual body temperature 205, x is the maximum one, a represents a proportional value, and b represents a compensation constant. Wherein, a is an average value for the proportions of the maximum temperatures and the actual body temperatures 205 measured by many times. Wherein, b is an average value for the differences of the maximum temperatures and the actual body temperatures 205 measured by many times.

In another embodiment, the second operation unit 224 may use the maximum temperature in each body temperature, but not limited to, to perform a non-linear compensation operation, including: \sqrt{y}=ax²+bx+c, wherein y is an actual body temperature 205, x is the maximum one, a, b represent proportional values, and c represents a compensation constant. Wherein, a and b are two average values for the proportions of the maximum temperatures and the actual body temperatures 205 measured by many times. Wherein, c is an average value for the differences of the maximum temperatures and the actual body temperatures 205 measured by many times.

Thereby, the system for detecting body temperature with temperature compensation provided by the present invention can detect several body temperatures around 2 to 3 meters, avoid interference from other non-human heat sources, and continuously perform compensation to the ambient temperature at that time automatically, so that the body surface temperature of the subject is closer to the actual temperature after compensation and the accuracy of the temperature measurement is also improved.

Looking at the above, the ambient temperature and/or the low temperature of the outdoor environment do/does influence the surface temperature of the human body and pull(s) down the surface temperature. Table 1 shows clear results. For real measurements, the surface temperatures detected by the forehead thermometer (surface) and the system of the present invention and viewed by thermal images are all below 30 degrees Celsius. Moreover, the temperature estimated by the forehead thermometer with temperature simulation is not satisfactory as well, it is not reasonable to be use as the core body temperature. On the contrary, the measurement of the system of the present invention compares the surface measurement result of the forehead thermometer at the low temperature of strong wind, for example, at the temperature of 11 degrees Celsius, the error within 2 degrees is maintained, and the numerical performance is stable and consistent with reference value.

TABLE 1
Measuring body temperatures at 11 degrees Celsius

| | | Forehead | | |
|---|---|---|---|---|
| | | thermometer | Forehead | |
| | Ear | (temperature | thermometer | This |
| Instrument | thermometer | simulation) | (surface) | invention |
| Temperature | 36.4 | 32.2 | 27.7 | 29 |
| (Celsius) | | | | |

The above embodiments are only used to illustrate the principles of the present invention, and they should not be construed as to limit the present invention in any way. The above embodiments can be modified by those with ordinary skill in the art without departing from the scope of the present invention as defined in the following appended claims.

What is claimed is:

1. A system for detecting body temperature with temperature compensation, the system comprising:
a first sensor, detecting a plurality of objects in an area and performing an identifying procedure, wherein the identifying procedure comprises facial recognition;
a second sensor, measuring temperatures of the plurality of objects and outputting a plurality of ambient temperatures and a plurality of body temperatures, wherein the plurality of ambient temperatures and the plurality of body temperatures are identified as non-body temperatures and body temperatures by the identifying procedure; and
a central processing unit, receiving and averaging the plurality of ambient temperatures to produce a current temperature, reading a reference temperature, comparing the current temperature with the reference temperature to produce a compensation, and performing operations for the compensation and the plurality of body temperatures to output a plurality of actual body temperatures.

2. The system according to claim 1, wherein the first sensor comprises an RGB thermal camera.

3. The system according to claim 1, wherein the area comprises 2 to 3 meters.

4. The system according to claim 1, wherein the identifying procedure comprises iris recognition.

5. The system according to claim 1, the second sensor comprises a non-contact temperature detecting sensor.

6. The system according to claim 1, wherein each of the plurality of body temperatures further includes a plurality of temperatures, the central processing unit only takes a maximum one in the each of the plurality of temperatures to operate with the compensation.

7. The system according to claim 1, wherein the reference temperature comprises a temperature-set corrected value.

8. The system according to claim 1, wherein each of the plurality of body temperatures further includes a plurality of temperatures, the central processing unit only takes a maximum one in each of the plurality of temperatures to perform a linear compensation operation, comprising: y=ax+b, wherein y is an actual body temperature, x is the maximum one, a represents a proportional value, and b represents a compensation constant.

9. The system according to claim 1, wherein each of the plurality of body temperatures further includes a plurality of temperatures, the central processing unit only takes a maximum one in each of the plurality of temperatures to perform a non-linear compensation operation, comprising: \sqrt{y}=ax²+bx+c, wherein y is an actual body temperature, x is the maximum one, a, b represent proportional values, and c represents a compensation constant.
